(19) United States
(12) Patent Application Publication
(10) Pub. No.: US 2004/0030264 A1
(43) Pub. Date: Feb. 12, 2004

(54) Method and apparatus for analyzing mammary gland fluid

(75) Inventor: David Hung, Redwood City, CA (US)

Correspondence Address: BANNER & WITCOFF, 1001 G STREET N W, SUITE 1100, WASHINGTON, DC, 20001, US

(73) Assignee: Cytyc Health Corporation, Boxborough, MA

(21) Appl. No.: 10/391,527

(22) Filed: Mar. 19, 2003

Related U.S. Application Data
(60) Provisional application No. 60/365,162, filed on Mar. 19, 2002.

(51) Int. Cl. A61B 5/00

(52) U.S. Cl. 600/573

(57) ABSTRACT

The present invention relates to an apparatus and method for collecting and analyzing a biological specimen such as ductal fluid from a mammary gland duct. The biological specimen is contacted with a solid phase and cell markers that may be indicative of breast cancer or other lesions may be identified. Early diagnosis of breast cancer may be achieved as well as therapeutic or prognostic factors may be identified.

### RELATED APPLICATIONS

This application claims benefit under 37 CFR §1.78 of provisional application 60/365,162 filed Mar. 19, 2002. The full disclosure of the application is incorporated hereby by reference.

### TECHNICAL FIELD

The present invention relates to a method and apparatus for analyzing mammary gland fluid and in particular to a method and apparatus for analyzing mammary gland fluid for managing or diagnosing mammary gland conditions.

### BACKGROUND OF THE INVENTION.

Breast cancer is a major cause of death in women. It is estimated that up to 10% of women in the United States are at risk of developing breast cancer in their lifetime. Methods of early detection have been developed such as physical examinations, regular self-examinations, mammography or tissue biopsy, however, inherent features of these methods limit their utility. Physical examinations and self-examinations may depend on the skill of the examiner and some lesions, particularly small-sized lesions, may be overlooked. Mammograms may sometimes be difficult to interpret in more dense breast tissue. Furthermore, mammograms may lack optimal sensitivity such that breast lesions may be present for many years and may develop to an advanced stage of disease before they are detectable on mammogram. Some breast tumors may grow undetected in breast tissue in excess of ten years before being detected by physical examination or mammography. Therefore, because advanced stage disease often carries a poor prognosis, reliance on mammogram may be less than optimal.

The use of cancer markers for early detection has been used. Markers may be cell surface or secreted proteins or nucleic acid sequences, for example, that may be formed by cancer cells. Numerous breast cancer cell markers have been identified which provide not only diagnostic information but also prognostic or treatment information. Cell marker studies may reveal information regarding the presence of tumor, tumor growth, invasion, metastatic potential, tumorigenesis, likelihood of response to a particular therapeutic option or may be used to track the progress of a course of therapy. Examples of breast cancer cell markers that have been identified include estrogen receptor (ER), progesterone receptor (PR), pS2, cathepsin D, hyaluronic acid (HA), tissue-type plasminogen activator (t-PA), erbB2 oncoprotein, epidermal growth factor receptor (EGR), CD44v5, CD44v6, p53, or Ki67, to name a few.

Evaluation of cell markers of breast tumor cells requires collecting a biological sample containing the cells. Traditionally, this required performing a tissue biopsy, which is both invasive and inconvenient to the patient. Moreover, tissue biopsy often requires a palpable lesion before sampling may be performed effectively at which time the lesion may have progressed to an advanced stage and may carry a poorer prognosis. If the lesion is more advanced, there may be a higher risk of treatment failure. This problem may be offset if the lesion could be detected earlier. Further, tissue biopsies often require anesthesia and concomitant surgical risks. Alternatively, fine needle aspiration is often performed where a needle is inserted into the suspicious lesion and contents are aspirated. However, this technique is often painful for the patient and may cause local reactions at the site of the biopsy such as scarring or inflammation that may make it difficult to visualize the tumor both grossly or microscopically. In such a situation, proper diagnosis is hampered and any tumor present may be missed. Also, if a tumor was present, the needle may cause spread of the tumor or cause artifactual changes during later histological review of the lesion. Therefore, effective non-invasive techniques in management of breast cancer is needed.

As a less invasive technique for breast cancer diagnosis, examination of nipple discharge or nipple aspirate fluid has been employed. These techniques are used in breast tumor analysis, especially for non-palpable lesions, and do not disturb the histology of breast tissue. However, it is extremely difficult to obtain a sample of adequate volume from the nipple to make an accurate diagnosis. If the sample is scant and no tumor cells are identified, it is not clear if the absence of tumor cells in the sample is due to the absence of tumor or due to the inadequacy of the obtained specimen as a result of using the nipple aspirating technique. Only certain types of tumors are believed to cause secretion of any appreciable amount of nipple discharge and those that cause such a secretion often produce less than 10 μl of nipple discharge fluid. This is often inadequate to make a definitive assessment.

Therefore, there exists a need in the art for non-invasive techniques for obtaining adequate breast fluid samples for early diagnosing and managing of breast cancer. A need also exists for performing cell marker studies on the obtained breast fluid samples for management, diagnosis, and analysis of breast lesions.

### SUMMARY OF THE INVENTION

The present invention relates to an apparatus and method for collecting a biological sample from a mammary gland duct comprising a collecting catheter capable of being inserted into a mammary gland duct, a chamber connected to the collecting catheter and comprising a solid phase, a pressure port and a valve interposed between the chamber and the pressure port. Cell markers within the biological sample are adsorbed onto the solid phase or onto binding agents that bind to the solid phase, for example. Analysis of the biological sample and/or cell markers provide an early diagnosis of breast cancer or other breast lesions as well as therapeutic options and prognostic factors.

### BRIEF DESCRIPTION OF THE DRAWINGS

FIG. 1 illustrates an exemplary apparatus for obtaining biological samples for analysis or assay of the present invention.

FIG. 2 illustrates another embodiment of a biological sample collection device and valve of the present invention.

FIGS. 3a-3c illustrate exemplary embodiments of a solid phase and valves of the present invention.

FIG. 4 illustrates another exemplary apparatus for obtaining biological samples for analysis or assay from a breast duct system involving a single port.

FIG. 5 illustrates another exemplary apparatus for obtaining biological samples for analysis or assay from a breast duct system involving Y-tube-shaped catheter.

### DETAILED DESCRIPTION OF THE INVENTION

The present invention provides a method and apparatus for collecting a biological sample from the breast non-invasively. The apparatus of the present invention may be introduced into a breast duct system and fluid from the breast duct system may be obtained and analyzed. The fluid obtained from the breast duct system may contact a solid phase that traps cell markers in the fluid. The cell markers may be indicative of the presence of tumors and include estrogen receptor (ER), progesterone receptor (PR), pS2, cathepsin D, hyaluronic acid (HA), tissue-type plasminogen activator (t-PA), erbB2 oncoprotein, epidermal growth factor receptor (EGR), CD44v5, CD44v6, p53, or Ki67, to name a few. Breast lesions may be identified early in their formation even before they are grossly visible or palpable.

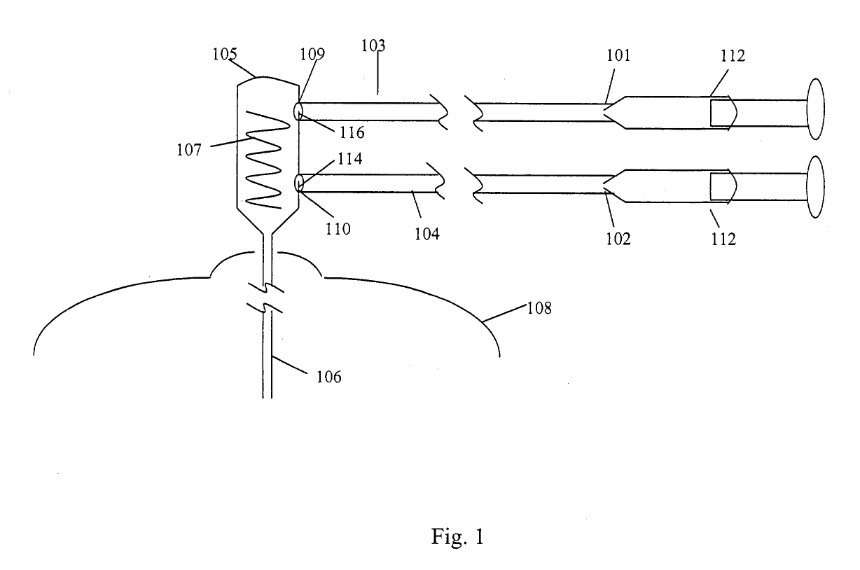

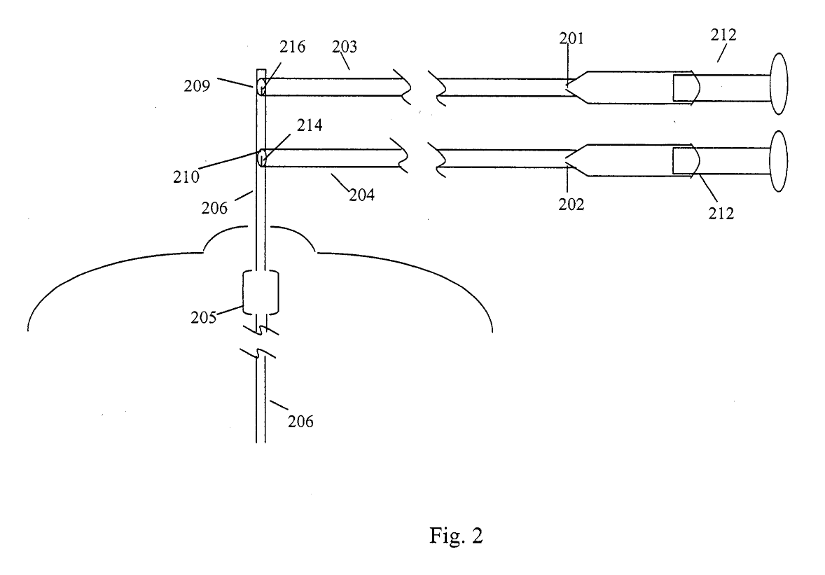

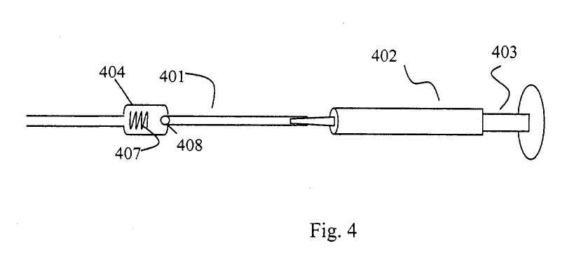

FIG. 1 illustrates one exemplary embodiment of the apparatus of the present invention for obtaining a biological sample from a breast duct system non-invasively in which multiple ports are utilized. FIG. 2 illustrates another exemplary embodiment of the apparatus of the present invention in which a main chamber is within a breast duct. FIG. 4 illustrates an alternative embodiment of a device for administering agents in which a single port is utilized. It should be noted that the illustrated devices are for illustration purposes only and they are not meant to limit the present invention as many similar devices may be utilized by a skilled artisan without departing from the scope or spirit of the invention.

As exemplified in FIG. 1, which demonstrates an illustrative embodiment of an apparatus 100 of the present invention, the exemplary apparatus for obtaining a biological sample from a breast duct contains an access device for accessing a breast duct such as a catheter or cannula that may be inserted into the breast duct. A biological sample may be obtained from the breast duct system via this apparatus or a separate catheter or cannula. U.S. patent application Ser. No. 09/473,510, David Hung, et al., filed Dec. 28, 1999, which is incorporated herein in its entirety, discloses an exemplary apparatus having an elongated ductal access device that can be used with the present invention for positioning within a breast duct.

FIG. 1 illustrates one exemplary embodiment of the apparatus 100 of the present invention. The apparatus 100 contains a contains a hollow elongated member with an internal lumen which can include a catheter or a cannula having an internal lumen extending between its ends (e.g., a catheter 106) for positioning within a breast duct and a main chamber or manifold 105 in fluid communication with the catheter 106. The main chamber 105 has an internal volume and an internal diameter that is greater than that of the catheter 106. The main chamber 105 also includes a first port 110 and a second port 109. These ports 109, 110 can be placed at any position discussed in U.S. patent application Ser. No. 09/473,510. For example, the second port 109 can be placed at the terminal end of the main chamber 105 and inline with the catheter 106. Additionally, the first port 110 can be positioned as close to catheter 106 as possible. Moreover, these ports 109, 110 can be vertically aligned with each other along the wall of the main chamber 105 or offset around the circumference of the main chamber 105.

Fluids and other materials can be introduced into and removed from the main chamber 105 through either of the illustrated ports 109, 110. As illustrated in FIG. 1, the first port 110 is connected to a first conduit 104 that has a port 102 for receiving an instrument such as a syringe 112. The second port 109 is connected to a second conduit 103 that has a port 101 for receiving a syringe 112. The syringes 112 can be replaced by any known collection and/or infusion device.

As discussed above, the port 102 and conduit 104 can be used to infuse a fluid into the main chamber 105 and into the duct via the catheter 106. In this case, ductal wash fluid, such as normal saline, is placed into the first port 102 and positive pressure is exerted at the first port 102 to expel the ductal wash fluid into the main chamber 105 and into the breast duct system via the catheter 106. Alternatively, ductal wash fluid may be placed into the second port 101 and expelled into the main chamber 105 through the second conduit 103 by exerting positive pressure at the second port 101. The ductal wash fluid may thus be administered into the chosen breast duct.

As previously mentioned, the port 101 and the conduit 103 can be used to collect material received from the duct and contained in the main chamber 105. For example, negative pressure may be exerted at the first port 109 by the operation of the syringe 112 connected to the port 101. This action produces a negative pressure in the main chamber 105 and draws the material obtained from the breast duct and residing in the main chamber 105 into the conduit 103 and the syringe 112 or other collection device.

The above descriptions of which ports and conduits are used to introduce fluid into the duct and collect material from within the duct are merely exemplary. Either set of conduits and ports can be used to perform either of these functions. Extraction of biological material which can include ductal fluids, cells (cell clumps) and the ductal wash fluid from the breast duct system may be accomplished by externally massaging the breast after the ductal wash fluid has been introduced into the duct. Additionally, negative pressure within the main chamber 105 can be caused by the operation of one or more of these syringes 112.

As shown in FIG. 1, valves 114, 116 may regulate the flow of material or fluid into and out of the main chamber 105 through the input port 110 and output port 109, respectively. The catheter 106 may have an internal lumen of a diameter sufficiently sized such that insertion into a breast duct system is facilitated while permitting the passage of desired agents and material. The catheter 106 may, for example, have a lumen diameter of 0.007 inches (or 0.178 mm) or greater, or a lumen diameter in the range from 0.007 inches (or 0.178 mm) to 0.047 inches (or 1.19 mm). Further, the catheter 106 may contain indicia on its surface to indicate the depth of insertion such that a user may be fully aware of the depth of insertion of the catheter 106 during insertion of the catheter 106 into the breast duct. Further, the catheter 106 may contain a safety mechanism such as a stop element such that the catheter 106 may not be further advanced into the breast duct system after a certain depth is attained. Such a stop element may be variously designed but may comprise, for example, a collar affixed to or formed on an exterior surface of the catheter 106, the collar being of a width greater than the diameter of the catheter 106.

As shown in FIG. 1, a solid phase 107 may be positioned within the main chamber 105. The solid phase 107 includes a fixed matrix on which cells containing a desired marker are immobilized and fills the main chamber 105. Alternatively, the solid phase 107 may partially fill the main chamber 105. Material passing into the main chamber 105 from the catheter 106 contacts the solid phase 107 prior to entering the conduits 103 or 104. Thus, for example, tumor cells from the ductal fluid adhere to the solid phase 107 as the ductal fluid enters the main chamber 105 and comes into contact with the solid phase 107. The solid phase 107 may also be used to trap expressed proteins or nucleic acid of interest. The solid phase can have any shape (for example round or the same as the cross section of the main chamber) that allows the collected fluid to pass through it between ports 101 and 102. It should be noted that the form of the solid phase 107 as well as the type of valve (114 or 116) may vary and are not limited by the exemplary embodiments illustrated in FIG. 1 or FIG. 2.

There are many ways in which to effect trapping of cells, proteins, nucleic acid, etc. onto the solid phase 107. For example, if cells expressing a particular cell surface marker are desired, the solid phase 107 may contain a reagent immobilized on its surface that has specificity for the cell marker. The reagent may be, for example, primary antibodies covalently bound to the solid phase 107 with binding specificity for the cell surface marker. In this case, as ductal fluid containing tumor cells expressing the cell surface marker contacts the reagent or antibodies that are immobilized on the solid phase 107, the cells are pulled out of the fluid, bind to the reagent and form an immobilized antigen-antibody complex. Likewise, if a protein expressed as a breast cancer marker is being assayed, reagents such as antibodies that bind specifically to the proteins may be immobilized onto the solid phase 107 in a similar manner.

In an alternate method of specimen collection, the ductal fluid received from the duct may be contacted with a reagent or ligand such as a polyclonal or monoclonal antibody specific for a protein breast cancer marker. A ligand-marker protein is thus formed in which the ligand is then immobilized onto the solid phase 107. In this example, ductal fluid may be received into the main chamber 105 and a ligand may be introduced into the main chamber 105 through the port 102 and first conduit 104. The ligand may bind to cell marker proteins present in the breast fluid in the main chamber 105 and the complex thus formed binds through the ligand to the solid phase 107. Binding may be accomplished by a variety of ways including but not limited to adsorption onto the matrix or binding through secondary binding partners, for example.

Detection of the cell marker protein present may be accomplished through a variety of methods. For example, labeled reagents may be used such as an anti-antibody that is coupled to a detectable agent. The detectable agent may be, for example, a chemical moiety such as a fluorescer, chemiluminescer, radioisotope or enzyme. Depending on the label used, detection of the presence of the cell marker may be accomplished by noting a change in properties after reaction with the label. Such changes include color changes, for example.

In another method of specimen collection, a cell marker protein is bound to a ligand. The ligand may have an affinity for a binding partner bound to the solid phase 107. As an example, the cell marker protein may bind to biotin as the ligand. Biotin may then bind to avidin, a binding partner immobilized to the solid phase 107. The ligand-marker protein thus binds to the solid phase through the binding partner. In this example, breast duct fluid may be aspirated into the main chamber 105 through the catheter 106. A ligand such as biotin may be introduced into the main chamber 105 through the port 102 and first conduit 104 to bind to the cell marker protein if present in the aspirated fluid. The biotin-marker protein complex may then bind to avidin that is immobilized to the solid phase 107.

Latex agglutination methods may also be used for specimen collection. In this example, particles may be coupled with another binding partner, then contacted with the breast fluid. Agglutination occurs due to formation of antibody linkages between the particles and detection of the agglutination may be determined by measuring the turbidity of the fluid, for example.

In another method, coating reagents are adsorbed onto the solid phase 107 which may be, for example antibodies or an affinity reagent such as avidin or streptavidin. The protein marker may be contacted by a primary antibody specific for the protein marker or may be crosslinked with a reagent to form a complex. This complex is then adsorbed onto the coating reagents on the solid phase 107. In this example, ductal fluid may be aspirated or forced under positive pressure applied to the breast such that ductal fluid enters into the main chamber 105, the ductal fluid containing a protein marker that may indicate the presence of breast cancer, for example. Antibodies specific for the protein marker are introduced into the main chamber 105 through the port 102 and the first conduit 104 or, alternatively, through the port 101 and the second conduit 103. The antibodies thus introduced bind with the protein marker present in the main chamber 105. Alternatively, a crosslinking reagent may be introduced into the main chamber 105 through the port 102 and first conduit 104 or the port 101 and the second conduit 103 and the crosslinking reagent may bind to the protein marker. The protein marker complex formed may then bind to coating reagents present on the solid phase 107.

The solid phase 107 may be made of many different types of materials such as but not limited to Sepharose, Protein A, Protein G, membranes, filters, pads, etc. A filter or membrane as the solid phase 107 may be contacted with the ductal fluid from the breast directly to facilitate the management of the collected specimen. For example, nitrocellulose may be utilized such that the marker protein collected on its surface may be then processed for visualization of the presence of the marker protein as well as analysis of the marker protein.

Another aspect of the invention involves collection of the biological specimen from the breast duct. In the exemplary embodiment described, the catheter 106 is inserted into a breast duct and ductal fluid is aspirated or forced under positive pressure applied to the breast such that the ductal fluid enters into a main chamber 105 connected to the catheter 106, the main chamber 105 being located external to the breast 108. However, the invention is not so limited as the apparatus may have many forms. For example, in another exemplary embodiment, the main chamber 105 may be inserted into the breast duct as discussed-below with respect to FIG. 2. In this embodiment, fluid is aspirated or forced under positive pressure applied to the breast into the main chamber 105 and the ductal fluid remains within the system. After reacting with the solid phase 107 in the main chamber 105, the fluid may be returned to the breast duct and the markers thus bound onto the solid phase 107 may then be analyzed.

In yet another embodiment, illustrated in FIG. 2, a main chamber 205 is positioned within the breast duct. Fluid may be aspirated from the breast duct system into the main chamber 205 through a ductal access device such as a catheter 206, by exerting positive pressure on the breast and/or negative pressure at the second port 201, for example. The solid phase (not illustrated) may be situated within the main chamber 205 as discussed above. As described, an appropriate cell marker may bind to the solid phase such that diagnostic, therapeutic or prognostic factors, for example, may be assessed.

In this example, fluid may be removed from the breast duct for further analysis if desired, however, fluid need not be removed from the breast duct. A valve structure 214 may be interposed between the first port 202 and the main chamber 205 and a valve structure 216 may be interposed between the second port 201 and the main chamber 205. The valve structures (214, 216) may be any known type and may be configured to prevent backflow of fluid into the first port 202 and second port 201, respectively. For example, a one-way valve 214 may be situated such that positive pressure exerted at the first port 202 may cause the valve to open and allow passage of material into the main chamber 205. Also, positive pressure applied to the breast may cause material to enter the main chamber 205 through the catheter 206, however, when the material such as ductal fluid fills the main chamber 205, the pressure within the main chamber 205 increases to a critical level such that the valve 214 at the first port 202 closes and prevents flow of fluid or material into the first port 202. If positive pressure is then exerted at the first port 202, pressure increases in the main chamber 205 over the pressure within the breast duct and the material or fluid flows from the main chamber 205 and back into the breast duct while positive pressure is applied to valve 216 from within the conduit 203. Similarly, the second port 201 and the corresponding valve 216 functions in a similar manner. Fluid and material from the breast duct, for example, may be aspirated into the main chamber 205 by exerting negative pressure at the second port 201 or by exerting positive pressure on the breast. As fluid and material fill the main chamber 205 from the breast duct and the pressure within the main chamber 205 rises, the pressure within the main chamber 205 reaches a critical level such that the pressure within the main chamber 205 rises above the pressure in the second port 201. At this time, the valve 216 remains open to allow flow into the second port 201 while valve 214 is closed to prevent backflow of fluid into the first port 201 and into a conduit 204. In this way, fluid may be concentrated in the main chamber 205 repetitively for improved specimen sampling.

It will be appreciated that the disclosed exemplary embodiments of an apparatus for administering ductal wash fluid into a breast duct system and for obtaining a biological specimen from the breast duct system are for illustration purposes only and are not intended to limit the present invention. Any suitable device suitable for injecting or infusing fluid into a duct or collecting biological material from the breast duct system may be utilized without deviating from the scope or spirit of the present invention.

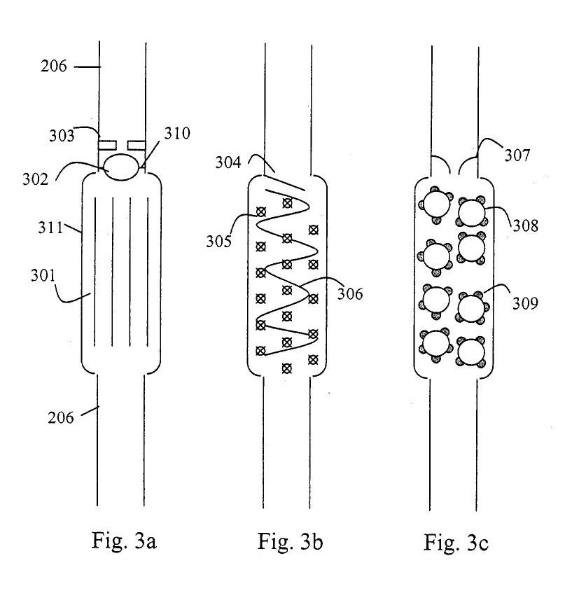

FIGS. 3a-3c illustrate examples of the solid phase of FIG. 2. The solid phase embodiments illustrated in FIGS. 3a-3c may be incorporated into the main cavity 205 of FIG. 2, for example. Alternatively, the embodiments of FIGS. 3a-3c may also be used as the solid phase 107 in the system illustrated in FIG. 1 such that the solid phase 107 may be contained within the main cavity 105 of FIG. 1. FIG. 3a illustrates a ball-type valve 302 attached to the internal surface of the catheter 106 by a flexible member 310 at a proximal end of an in-line chamber 311 containing the solid phase 301. A cuff 303 is also provided adjacent to and on the proximal side of the ball-type valve 302 such that a seal may be formed depending on the pressure exerted on the ball-type valve 302. When pressure is applied to the ball-type valve 302, the ball-type valve 302 moves in the direction of lower pressure. If pressure is exerted through the cuff 303, for example if material is passed into the in-line chamber 311 from the proximal end, the ball-type valve 302 is forced away from the cuff 303 and material may thus flow through the catheter 106. If the pressure is higher distal to the ball-type valve 302, however, the ball-type valve 302 may be pressed against the cuff 303, thus closing the opening and preventing flow of material beyond the ball-type valve. In this way, fluid may pass in one direction but not in the other. FIG. 3b illustrates a second exemplary embodiment of a valve. In this example, the valve comprises a flat hinged member 304 such that pressure in one direction causes the flat hinged member 304 to open while pressure in the opposite direction causes the flat hinged member 304 to close and prevent further flow of material out of the in-line chamber 311 and through the valve. FIG. 3c illustrates another exemplary embodiment wherein the valve comprises a plurality of contoured valve leaflets 307 such that pressure exerted, for example, if material is forced through the catheter 106 and into the in-line chamber 311, this pressure causes the ends of the contoured valve leaflets 307 to move in the direction of the lower pressure such that the ends of the contoured valve leaflets 307 become unapposed. This enables the passage of material into the in-line chamber 311. Conversely, if the pressure distal to the contoured valve leaflets 307 is greater than the pressure proximal to the contoured valve leaflets 307, such as when material is being passed from the in-line chamber 311 through the contoured valve leaflets 307 and into the catheter 106, the contoured valve leaflets 307 become apposed and a seal develops such that material is prevented from passing through the contoured valve leaflets 307.

FIGS. 3a-3c also illustrate various examples of the solid phase in the main cavity 205 of FIG. 2. Alternatively, the solid phase embodiments illustrated in FIGS. 3a-3c may be incorporated into the system illustrated in FIG. 1 such that the solid phase 107 is contained in the main cavity 105. FIG. 3a illustrates a flat solid phase 301 within the inline chamber 311. When material is drawn into the in-line chamber 311, the material contacts the flat solid phase 301. As described below, the flat solid phase 301 may contain reagents bound thereto. FIG. 3b illustrates a variation of the solid phase in which a flat and curved solid phase 306 is used. When material contacts the flat and curved solid phase 306, material with an affinity for bound reagents on the flat and curved solid phase 306 may bind. The flat and curved shape provides increased surface area for binding. FIG. 3b further illustrates an alternative to binding of a compound to the solid phase wherein the compound binds to a ligand that has an affinity for a binding partner bound to the solid phase 306. The compound binds to the ligand forming a compound-ligand complex 305 which in turn may bind to the solid phase. This process is described in more detail below. FIG. 3c illustrates another form of a solid phase wherein a bead-type solid phase 308 is used. In this example, the bead-type solid phase 308 comprises a solid matrix in the form a bead. The bead-type solid phase 308 contains a reagent 309 bound to its surface. This reagent 309 may be, for example, an antibody with an affinity for a desired compound. The bead-type solid phase may be made of a variety of materials, such as but not limited to agarose, sephadex, cellulose, polymers, etc. These processes are herein in exemplary embodiments. It should be noted that the examples provided of the valve and the solid phase are merely exemplary and are not intended to limit the present invention as any similar valve or solid phase may be used. In another embodiment, the solid phase 107 can be positioned between the port 102 and the catheter 106 and have one of the forms previously discussed.

FIG. 4 illustrates another exemplary embodiment of an apparatus for obtaining biological material from a breast duct system or administering breast ductal wash fluid into a breast duct system. In this embodiment, the apparatus is a single lumen device comprising a ductal access device such as a catheter 401 in connection with a syringe 402. The ductal access device or catheter 401, for example, may contain an in-line chamber 404 containing a solid phase 407, similar to those discussed above, and a valve 408 as described. The syringe 402 enables introduction of ductal wash fluid into the breast duct system or extraction of biological material or breast ductal wash fluid, such as normal saline, from the breast duct system. A plunger 403 may be situated at a top end of the syringe 402, for example, and may be used to introduce ductal wash fluid contained within the syringe 402 by exerting pressure at the plunger 403 into the breast duct system (not shown) and be used to withdraw biological material from the breast duct system when external pressure is applied to the breast. The collection can also be assisted by exerting negative pressure at the plunger 402. In this way, material may pass through the in-line chamber 404 and contact the solid phase 407 as described with the valve 408 regulating the flow of the material. Alternatively, the lumen of the syringe may contain the solid phase 407 or an additional solid phase (not shown), which may be a fixed matrix on which cells containing a desired marker are immobilized. Thus, tumor cells from the ductal fluid adhere to the solid phase 407 as the ductal fluid enters the lumen of the syringe 402 and comes into contact with the solid phase 407. The solid phase 407 may also be used to trap expressed proteins or nucleic acid of interest. The in-line chamber 404 and valve 408 may be omitted in this embodiment (not shown).

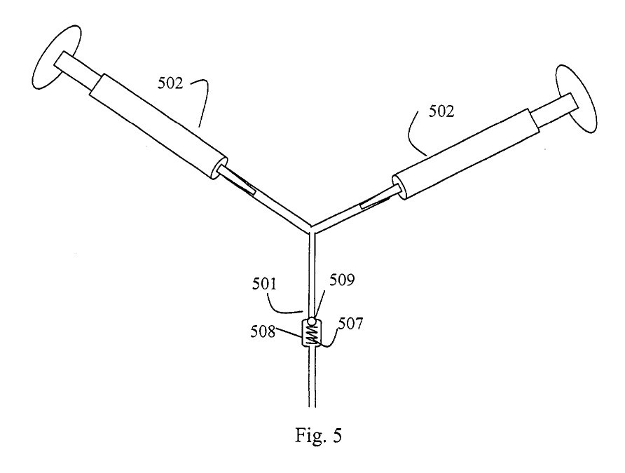

FIG. 5 illustrates another exemplary embodiment of an apparatus for administering ductal wash fluid into a breast duct system or obtaining biological material or ductal wash fluid, such as normal saline, from the breast duct system. In this embodiment, a syringe 502 is connected to a Y-tube catheter 501 at each of a plurality of proximal ends of which two are shown. The distal end of the Y-tube-shaped catheter may be inserted into a breast duct system via a nipple surface. The proximal ends of the Y-tube catheter 501 may contain ports 502 for administering ductal wash fluid or collecting biological material from the breast duct system. Ductal wash fluid, such as normal saline, may be introduced into the breast duct system from any of the plurality of proximal ends of the Y-tube catheter 501. Alternatively, biological material or administered ductal wash fluid may be extracted from the breast duct system when external pressure is applied to the breast as discussed above with respect to the other embodiments. Collection of the biological material can also be aided by exerting negative pressure at any of the ports of the Y-tube-shaped catheter 501. Material may pass through an in-line chamber 508, the in-line chamber 508 containing a solid phase 507 and a valve 509 to regulate flow as described such that the material contacts the solid phase 507 within the in-line chamber 508. FIG. 5 illustrates the solid phase 507 in the in-line chamber 508 of the Y-tube catheter 501. Alternatively, the in-line chamber 508 is not used and the lumen of the Y-tube catheter 501 or the lumen of a syringe 502 may contain a solid phase 507. In either embodiment, the solid phase 507 may be a fixed matrix on which cells containing a desired marker are immobilized. Thus, tumor cells from the ductal fluid adhere to the solid phase 507 as the ductal fluid enters the in-line chamber 508 of the Y-tube catheter 501 and comes into contact with the solid phase 507. The solid phase 507 may also be used to trap expressed proteins or nucleic acid of interest.

Although the illustrative embodiments of the invention have been described, a wide range of modifications, changes and substitutions is intended in the foregoing disclosure. It is understood that the present invention can take many forms and embodiments. The embodiments shown herein are intended to illustrate rather than to limit the invention, it being appreciated that variations may be made without departing from the spirit of the scope of the invention.

What is claimed is:

1. An apparatus for collecting a biological sample from a mammary gland duct comprising:
a collecting catheter capable of being inserted into a mammary gland duct;
a chamber connected to the collecting catheter and comprising a solid phase; and
a pressure port.

2. The apparatus of claim 1 wherein the solid phase is selected from the group consisting of sepharose, protein A, protein C, membranes, filters, pads and nitrocellulose.

3. The apparatus of claim 1 wherein the solid phase comprises a reagent.

4. The apparatus of claim 3 wherein the reagent is adsorbed onto the surface of the solid phase.

5. The apparatus of claim 4 wherein the reagent comprises an antibody.

6. The apparatus of claim 5 wherein the antibody specifically binds an antigen selected from the group consisting of estrogen receptor (ER), progesterone receptor (PR), pS2,cathepsin D, hyaluronic acid (HA), tissue-type plasminogen activator (t-PA), erbB23 oncoprotein, epidermal growth factor receptor (EGR), CD44v5, CD44v6, p53, and Ki67.

7. The apparatus of claim 1 further comprising a valve interposed between the chamber and the pressure port.

8. The apparatus of claim 7 wherein the valve prevents flow of fluid from the main chamber to the pressure port.

9. The apparatus of claim 1 further comprising a second pressure port.

10. The apparatus of claim 9 wherein a reagent is introduced into the chamber via the second pressure port.

11. The apparatus of claim 1 wherein the chamber is capable of being inserted into a breast duct.
